(19) United States
(12) Patent Application Publication
(10) Pub. No.: US 2018/0028493 A1
(43) Pub. Date: Feb. 1, 2018

(54) INHIBITORS OF INDUCED MMP-1 PRODUCTION

(71) Applicants: Jeanine D'Armiento, New York, NY (US); Takayuki Shiomi, New York, NY (US)

(72) Inventors: Jeanine D'Armiento, New York, NY (US); Takayuki Shiomi, New York, NY (US)

(73) Assignee: THE TRUSTEES OF COLUMBIA UNIVERSITY IN THE CITY OF NEW YORK, New York, NY (US)

(21) Appl. No.: 15/675,361

(22) Filed: Aug. 11, 2017

Related U.S. Application Data
(63) Continuation-in-part of application No. PCT/US2016/017562, filed on Feb. 11, 2016.
(60) Provisional application No. 62/115,021, filed on Feb. 11, 2015.

(51) Int. Cl. A61K 31/366; A61K 31/381; A61K 31/496; A61K 31/138; G01N 33/50; A61K 31/47; A61K 31/405; A61K 31/4418; A61K 31/397; A61K 9/00; A61K 31/135

(52) U.S. Cl. CPC A61K 31/366; A61K 9/0073; A61K 31/381; A61K 31/496; A61K 31/138; A61K 31/135; A61K 31/47; A61K 31/405; A61K 31/4418; A61K 31/397; A61K 9/0014; G01N 33/5008; G01N 2333/96494; G01N 33/6893

(57) ABSTRACT

The present invention provides methods and compositions of prophylaxis for, or for treating, chronic obstructive pulmonary disease (COPD), cancer, arthritis, skin damage, or atherosclerotic plaque rupture in a subject in need thereof, which comprise i) a statin or ii) selective serotonin reuptake inhibitor (SSRI).

This application is a continuation-in-part of PCT International Application No. PCT/US2016/017562, filed Feb. 11, 2016, claiming the benefit of U.S. Provisional Application No. 62/115,021, filed Feb. 11, 2015, the contents of each of which are hereby incorporated by reference into the application.

This invention was made with government support under grant number R01HL086936 awarded by the National Institutes of Health. The U.S. Government has certain rights in the invention.

Throughout this application, various publications are referenced, including referenced in parentheses. Full citations for publications referenced in parentheses may be found listed at the end of the specification immediately preceding the claims. The disclosures of all referenced publications in their entireties are hereby incorporated by reference into this application in order to more fully describe the state of the art to which this invention pertains.

### BACKGROUND OF INVENTION

Chronic obstructive pulmonary disease (COPD) is an enormous unmet medical need. Present therapies offer relief from its symptoms, but no drug treats the cause or slows progression of the disease. The most common cause of COPD is cigarette smoking, a behavior whose prevalence in the U.S. has remained fairly constant but continues to rise worldwide. In the U.S. alone, each year this disease results in more than 100,000 deaths, is responsible for over 600,000 hospitalizations and over 15 million physician office visits, causing approximately 150 million days of disability (CDC 2003). It is estimated that about 600 million adults have COPD, of which 24 million live in the U.S. (CDC, 2003).

In 2010 the annual cost for COPD was $20.4 billion in direct health care expenditures, and $29.5 billion in indirect costs (COPD Fact Sheet, 2014). As of 2008 COPD became the third leading cause of death (Minino et al., 2011) and analysts estimate the worldwide market for COPD therapy at $15.6 billion in 2019 (GOLD, 2013). Spiriva® (tiotropium, Boehringer Ingelheim/Pfizer) was launched outside the U.S. in 2002 and is marketed exclusively for COPD. Its European sales are over 2.4 billion euros and US sales topping 1 billion (COPD Market to 2019, 2013). Recently, Roflumilast, a phosphodiesterase type 4 (PDE-4) inhibitor was approved as a new therapeutic for COPD exacerbations with sales progressively growing since release (Fabbri et al., 2010).

The massive health cost burden of COPD is due to a combination of an increased incidence and sub-optimal treatment strategies. In the past, the desire to develop new pharmaceuticals has met with resistance because targets have been difficult to select and test and, furthermore, the disease has been treated as “self-inflicted” by the public and has not therefore received the attention warranted by its human and economic costs. The industry has recently witnessed high-profile attitude changes, and therefore, today the present barrier to the creation of effective drugs for COPD is the development of agents that act upon validated drug targets in this disease (COPD Market to 2019, 2013).

More than 43.8 million, or 19%, of adults in the U.S were smokers in 2011 (CDC, 2011). While the prevalence of current smoking during 2005-2011 has been slightly declining overall (CDC, 2012), the worldwide prevalence of smoking continues to rise. Smokers are ten times more likely than non-smokers to die of COPD. Smoking cessation is the only intervention of proven value in early-stage COPD, however, even with cessation, the destructive process initiated by cigarette smoking continues (COPD Fact Sheet, 2014) emphasizing the need for therapies targeted towards smoke induced inflammation and lung destruction.

Present interventions used for COPD serve to ameliorate the symptoms of the disease but do not address its overall course. The physiologic hallmark of COPD is fixed airway obstruction with a progressive decline in the forced expiratory volume in one second (FEV1). Bronchodilators, including anticholinergics (e.g., Atrovent®, Spiriva®) and β-adrenergic agonists (e.g., albuterol, Opened®), relax airway smooth muscle and appear to decrease dyspnea, increase FEV1, and decrease the frequency of reported exacerbations in certain populations (Hanania and Marciniuk, 2011). The effect of bronchodilators is short-lived, however, and these agents do not slow the progression of the disease as measured by a long-term decline in FEV1 (Hanania and Marciniuk, 2011). The regular use of inhaled corticosteroids (e.g., Flovent®) reduces symptoms, frequency of exacerbations, and numbers of outpatient physician visits in patients with moderate or severe COPD, but does not affect the rate of decline in post-bronchodilator FEV1 (Hanania and Marciniuk, 2011). However, chronic use of systemic corticosteroids does not improve the course of COPD, and may increase mortality (Hanania and Marciniuk, 2011).

New methods and compositions for treating COPD are needed.

### SUMMARY OF THE INVENTION

The present invention provides a method of prophylaxis for, or for treating, chronic obstructive pulmonary disease (COPD), cancer, arthritis, skin damage, or atherosclerotic plaque rupture in a subject in need thereof, which comprises administering to the subject i) a statin or ii) a selective serotonin reuptake inhibitor (SSRI) in an amount that is effective to treat the COPD, cancer, arthritis, skin damage, or atherosclerotic plaque rupture.

The present invention also provides a composition for use in prophylaxis for, or in treating, chronic obstructive pulmonary disease (COPD), cancer, arthritis, skin damage, or atherosclerotic plaque rupture in a subject in need thereof which comprises i) a statin or ii) a selective serotonin reuptake inhibitor (SSRI).

Aspects of the present invention relate to the use of i) a statin or ii) a selective serotonin reuptake inhibitor (SSRI) for the manufacture of a medicament for the treatment of chronic obstructive pulmonary disease (COPD), cancer, arthritis, skin damage, or atherosclerotic plaque rupture in a subject in need thereof.

The present invention further provides an inhaler containing a statin or an SSRI.

### BRIEF DESCRIPTION OF THE DRAWINGS

FIG. 1. Cigarette smoke induces MMP-1 through a MAPKinase dependent pathway via a conserved cigarette smoke element (CRE) that induces the transcription of MMP-1 (Mercer et al., 2009). This CRE is conserved in several MMPs and cytokines. A library of compounds was screened for their ability to block the cigarette smoke induction of MMP-1. These compounds will then be tested for their ability to block the inflammatory cascade and induction of other MMPs. If successful these compounds will be further tested in vivo for their ability to protect from emphysema formation and further developed as a therapeutic in the disease.

FIG. 2A. Intra-assay variability test. To test for reproducibility of the assay, cells were transfected with the MMP-1/pGL3 reporter plasmid. Cells were seeded in three 96-well plates using an interleaved format and treated with, 5% CSE media (H), 1% CSE (M), No CSE (L). After 1, 2 and 3 days incubation, the luciferase activity was measured in each well.

FIG. 2B. Variability test with a known small inhibitor compound. To test for function of the assay, cells were transfected with the MMP-1/pGL3 reporter plasmid. Cells were seeded in three 96-well plates using an interleaved format and treated with, 5% CSE media with various concentrations of PD098059 (an ERK inhibitor) After 24 hr incubation, the luciferase activity was measured in each well.

FIG. 3. Inhibitory effect of a collection of 727 small molecule compunds on CSE induced MMP-1 transcription activity. For the fifteen compounds identified as active during the primary screen, independent compound batches were obtained from the NIH Molecular Libraries Small Molecule Repository and 10 dilution points, of 1:3 serial dilutions starting from a nominal 10 mM solution prepared and tested in triplicate for inhibition of CSE/MMP-1 induction. IC50 values were calculated for each compound using a four-parameter equation describing a sigmoidal dose response curve. Lead compounds were selected if they possessed an IC50 value ≦1 μM (Data not shown).

FIG. 4. Reduction of markers for inflammation after treatment of SAECs with CSE and Compound 1 (simvastatin, Class A-Statin). SAEC treatment with 5% CSE and 10 uM Compound 1 (simvastatin, a Statin), showing decreased IL-8 levels. Compound 1 (simvastatin, a Statin) decreases the induction of IL-8 after cigarette smoke treatment indicated by *). Data is presented as mean+standard error. There was no statistically significant difference in the expression of IL-8 between un-treated cells and those treated with compound 1 (simvastatin, a Statin) under non-smoke exposed conditions.

FIG. 5. Blockade of MMP-1 induction after treatment of SAECs with CSE and Duloxetine. SAEC treatment with 5% CSE and 10 uM Duloxetine, demonstrating decreased MMP-1 expression. Duloxetine decreases the induction of MMP-1 after cigarette smoke treatment indicated by * p<0.05. Data is presented as mean+standard error.

FIG. 6. The expression of MMP-1 protein in BAL from smoke rabbits. Duloxetine administrated to smoke exposed rabbits down regulated the expression of MMP-1 in BAL. SM-smoke without the duloxetine, SMD-smoke with duloxetine.

FIG. 7A. Attenuation of smoke induced emphysema in a rabbit smoke exposure model. H&E representation of lungs from rabbits untreated, treated with duloxetine, smoke exposed, and smoke exposed with duloxetine. Duloxetine was administered 3 mg/day given once a day. The development of emphysema was blocked in the treated group.

FIG. 7B. Morphometric analysis of rabbit study groups. SM group had statistically significant increased mean linear intercept (unit: μm) compared to the rest of the groups (p=0.0495). NS-non-smoke without the duloxetine, NS-D-non-smoke with duloxetine, SM-smoke without the duloxetine, SM-D-smoke with duloxetine.

FIG. 8. Blockade of MMP-1 induction after treatment of SAECs with CSE and Fluoxetine. SAEC treatment with 5% CSE, 10 nM Fluoxitine, 10 uM Fluoxitine, and combinations of 5% CSE and Fluoxitine. CSE induces MMP1 expression and when treated with Fluoxetine and CSE MMP-1 induction was attenuated (p<0.05). Data is presented as mean+standard error.

FIG. 9A. Cigarette Smoke induces TLR4 expression in SAE cells, which is blocked when cells are treated with Fluoxetine. SAEC treatment with 5% CSE and 10 uM fluoxetine (Fluo), exhibit down regulation of TLR-4 receptor expression.

FIG. 9B. Western blot analysis demonstrates that the phosphorylation of IRAK (downstream target of TLR-4 signaling pathway) was suppressed by Fluoxetine.

FIG. 10. Protein Array Analysis of Cells Treated with CSE compared to Cells Treated with CSE combined with Fluoxetine. Three transcription factors were found to increase with CSE treatment and when pretreated with Fluoxetine these factors return to baseline. PRAS40, BAD and GSK-3b are increased with cigarette smoke treatment and return to baseline upon Fluoxetine treatment. The lines representing these three transcription factors, along with ERK 1/2, are labeled. Additionally, the lines compressed at the bottom of the figure represent the following transcription factors that were also tested: PC, NC, Stat1, Stat2, Akt (Thr308), Akt (Ser473), AMPKa, S6 Ribosomal Protein, mTOR, HSP27, p70 S6 Kinase, p53, p38, SAPK/JNK, PARP and Caspase-3.

FIG. 11A. Standard Fluoxetine Chromatographic Characteristics. The chromatogram of Fluoxetine displays a retention time of 17.69 mins. The peak corresponding to Fluoxetine is highlighted.

FIG. 11B. Absorbance spectra for Fluoxetine.

FIG. 12A. Chromatographic Characteristics of Control Samples. The standard chromatogram for no drug administration.

FIG. 12B. The standard chromatogram for administration of PBS.

FIG. 13A. Fluoxetine Quantification in Oral Gavage Method of Drug Administration. A study was conducted to determine whether fluoxetine is absorbed in the lung when delivered orally. Animals were given PBS and 10 mg/kg oral gavage of fluoxetine. Mice were sacrificed, lungs lyophilized and protein homogenate prepared for chromatographic analysis. Oral gavage of fluoxetine yielded two compounds showing chromatographic peaks at similar retention times.

FIG. 13B. Absorbance spectra suggested the occurrence of a metabolite of fluoxetine, an isomer or enantiomer of the compound.

FIG. 14A. Fluoxetine Quantification in Inhalation Method of Drug Administration. A study was conducted to determine whether fluoxetine is absorbed in the lung when delivered through inhalation. Animals were given PBS and 10 mg/kg of inhaled Fluoxetine. Mice were sacrificed, lungs lyophilized and protein homogenate prepared for chromatographic analysis. Inhalation of fluoxetine yielded three compounds that can be identified in lung samples.

FIG. 14B. Absorbance spectra of the three peaks showed that two of the compounds have almost identical absorbance spectra; the third compound retained a more polar characteristic eluting at 14.7 mins.

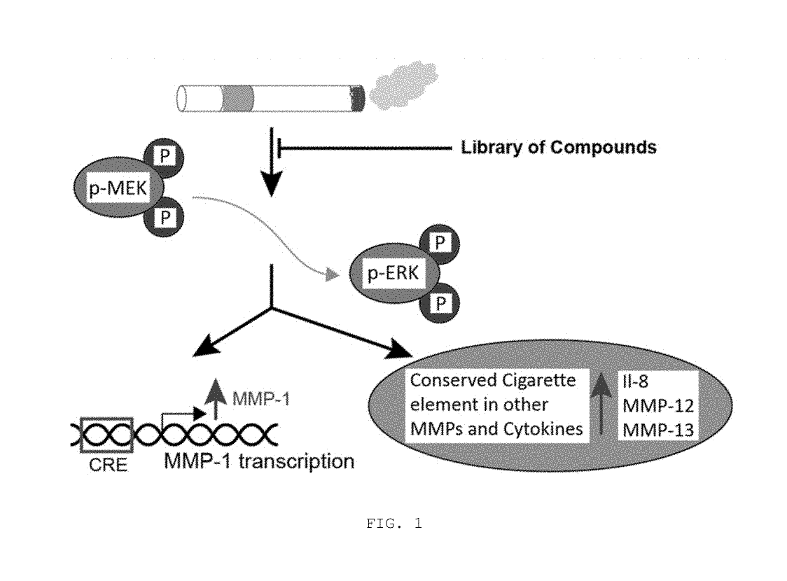

### DETAILED DESCRIPTION OF THE INVENTION

The present invention provides a method of prophylaxis for, or for treating, chronic obstructive pulmonary disease (COPD), cancer, arthritis, skin damage, or atherosclerotic plaque rupture in a subject in need thereof, which comprises administering to the subject i) a statin or ii) a selective serotonin reuptake inhibitor (SSRI) in an amount that is effective to treat the COPD, cancer, arthritis, skin damage, or atherosclerotic plaque rupture.

In some embodiments, the method is for treatment of a subject who has been diagnosed with COPD, cancer, arthritis, skin damage, or atherosclerotic plaque rupture.

In some embodiments, the method is for treating COPD.

In some embodiments, the method is for prophylactic treatment of a subject for COPD.

In some embodiments, the amount of the statin or the SSRI is effective to improve pulmonary function in the subject compared to i) the subject before administration of the amount of the statin or the SSRI or ii) a corresponding subject who has not been administered the amount of the statin or the SSRI.

In some embodiments, the amount of the statin or the SSRI is effective to reduce pulmonary inflammation in the subject compared to i) the subject before administration of the amount of the statin or the SSRI or ii) a corresponding subject who has not been administered the amount of the statin or the SSRI.

In some embodiments, reduced pulmonary inflammation in the subject comprises
a) reduced expression of at least one cytokine or
b) a reduced number of neutrophils in the lungs of the subject.

In some embodiments, reduced pulmonary inflammation in the subject comprises a reduced expression of interleukin 8 (IL-8) in the lungs of the subject.

In some embodiments, treating the subject comprises reducing the expression of at least one protease in the lungs of the subject compared to i) the subject before administration of the amount of the statin or the SSRI or ii) a corresponding subject who has not been administered the amount of the statin or the SSRI.

In some embodiments, the at least one protease is at least one matrix metalloproteinase (MMP).

In some embodiments, the at least one MMP comprises at least MMP-1, MMP-2, MMP-9, MMP-12 or MMP-13.

In some embodiments, the COPD comprises emphysema.

In some embodiments, the amount of the statin or the SSRI is effective to slow, halt, or reverse the progression of emphysema in the subject.

In some embodiments, the statin or the SSRI is capable of reducing cigarette smoke-induced or cigarette smoke extract (CSE)-induced MMP-1 expression without causing cytotoxicity.

In some embodiments, the statin or the SSRI is capable of reducing cigarette smoke-induced or CSE-induced MMP-1 expression with an IC50 equal to or less than 1 pM.

In some embodiments, the statin or the SSRI is capable of reducing cigarette smoke-induced or CSE-induced MMP-1 expression by 80-120%, wherein the level of MMP-1 expression in the absence of cigarette smoke or CSE induction is 100%.

In some embodiments, the statin or the SSRI is capable of reducing MMP-1 or IL-8 expression in small airway epithelial cells (SAECs) contacted with cigarette smoke or CSE.

In some embodiments, the statin or the SSRI is capable of reducing expression of TLR-4 receptor, PRAS40, BAD or GSK-3b, or reducing IRAK phosphorylation in SAECs in the subject.

In some embodiments, the expression is reduced by 80-120%, wherein the baseline level of expression is 100%.

In some embodiments, the statin or the SSRI is an organic compound having a molecular weight less than 1000 Daltons, a DNA aptamer, an RNA aptamer, or a polypeptide.

In some embodiments, the statin or the SSRI is an organic compound having a molecular weight less than 1000 Daltons.

In some embodiments, a statin is administered to the subject.

In some embodiments, the statin is Simvastatin, Lovastatin, Itavastatin, Fluvastatin, Mevastatin, Cerivastatin or Ezetimibe, or a pharmaceutically acceptable salt or ester thereof.

In some embodiments, the statin is a compound that
a) is in a clinical trial;
b) is approved for use in human subjects; or
c) was previously approved for use in human subjects but whose approval was subsequently withdrawn.

In some embodiments, an SSRI is administered to the subject.

In some embodiments, the SSRI is Duloxetine, Nefazodone, Fluoxetine or Sertraline, or a pharmaceutically acceptable salt or ester thereof.

In some embodiments, the SSRI is a compound that
a) is in a clinical trial;
b) is approved for use in human subjects; or
c) was previously approved for use in human subjects but whose approval was subsequently withdrawn.

In some embodiments, the subject is a mammalian subject.

In some embodiments, the subject is a human subject.

In some embodiments, the subject is or was a cigarette smoker.

In some embodiments, the COPD is caused by chronic cigarette smoking.

In some embodiments, the statin or the SSRI is administered to the subject as an aerosol.

In some embodiments, the statin or the SSRI is administered to the subject using an inhaler.

In some embodiments, the statin or the SSRI is administered to the subject in a dose of between 0.1 mg/kg to 2 mg/kg.

In some embodiments, the statin or the SSRI is administered to the subject in a dose of about 0.8 mg/kg.

In some embodiments, the method is for treating skin damage.

In some embodiments, administering the statin or the SSRI to the subject comprises topically applying the statin or the SSRI to the subject's skin.

In some embodiments, the amount of the statin or the SSRI is effective to reduce the expression of at least one cytokine or at least one protease in the subject compared to i) the subject before administration of the amount of the statin or the SSRI or ii) a corresponding subject who has not been administered the amount of the statin or the SSRI.

In some embodiments, the subject is
a) a COPD-drug, cancer-drug, arthritis-drug, skin damage-drug, or atherosclerotic plaque rupture-drug naïve subject;
b) a statin naïve subject; or
c) an SSRI naïve subject.

The present invention also provides a composition for use in prophylaxis for, or in treating, chronic obstructive pulmonary disease (COPD), cancer, arthritis, skin damage, or atherosclerotic plaque rupture in a subject in need thereof which comprises i) a statin or ii) a selective serotonin reuptake inhibitor (SSRI).

In some embodiments, the subject is
a) a COPD-drug, cancer-drug, arthritis-drug, skin damage-drug, or atherosclerotic plaque rupture-drug naïve subject;
b) a statin naïve subject; or
c) an SSRI naïve subject.

Aspects of the present invention relate to the use of i) a statin or ii) a selective serotonin reuptake inhibitor (SSRI) for the manufacture of a medicament for the treatment of chronic obstructive pulmonary disease (COPD), cancer, arthritis, skin damage, or atherosclerotic plaque rupture in a subject in need thereof.

In some embodiments, the treatment is prophylactic treatment.

In some embodiments, the subject is
a) a COPD-drug, cancer-drug, arthritis-drug, skin damage-drug, or atherosclerotic plaque rupture-drug naïve subject;
b) a statin naïve subject; or
c) an SSRI naïve subject.

The present invention further provides an inhaler containing a statin or an SSRI.

In some embodiments, the inhaler is for use in treating a subject afflicted with chronic obstructive pulmonary disease (COPD).

In some embodiments, the subject is
a) a COPD-drug, cancer-drug, arthritis-drug, skin damage-drug, or atherosclerotic plaque rupture-drug naïve subject;
b) a statin naïve subject; or
c) an SSRI naïve subject.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, “about” in the context of a numerical value or range means±10% of the numerical value or range recited or claimed, unless the context requires a more limited range.

As used herein, a subject “in need” of treatment for a disease, e.g. COPD, cancer, arthritis, skin damage, or atherosclerotic plaque rupture, means a subject who was been affirmatively diagnosed to have the disease.

As used herein, a subject who is “naïve” for a drug used to treat a disease is a subject who has not been administered any drug for that disease. Therefore, a COPD-drug naïve subject has not been administered any drug for COPD, a cancer-drug naïve subject has not been administered any drug for cancer, an arthritis-drug naïve subject has not been administered any drug for arthritis, a skin damage-drug naïve subject has not been administered any drug for skin damage, and an atherosclerotic plaque rupture-drug naïve subject has not been administered any drug for atherosclerotic plaque rupture.

As used herein, a “statin naïve subject” is a subject that has not been administered any statin.

As used herein, an “SSRI naïve subject” is a subject that has not been administered any SSRI.

As used herein, “effective” when referring to an amount of a compound or compounds refers to the quantity of the compound or compounds that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the physical condition of the patient, the type of subject being treated, the duration of the treatment, the nature of concurrent therapy (if any), and the specific formulations employed and the structure of the compound or its derivatives.

As used herein, “approved for use in human subjects” means approved for any medicinal use in human subjects at any time by any government agency of any country. In some embodiments, a compound that has been approved for use in human subjects was approved by the Food and Drug Administration (FDA) of the United States. For example, a statin that is approved for use in human subjects may in some embodiments be a statin that is approved for use in treating human subjects afflicted with high cholesterol or cardiovascular disease. Similarly, an SSRI that is approved for use in human subjects may in some embodiments be an SSRI that is approved for use in treating depression by the FDA.

Non-limiting examples of commercially available statins include: Simvastatin, Lovastatin, Itavastatin, Fluvastatin, Mevastatin, Cerivastatin and Ezetimibe.

Simvastatin is a statin that is commercially available from Merck Sharp & Dohme Corp. (Cramlington, Northumberland, UK NE23 3JU). The CAS Registry number for Simvastatin is 79902-63-9. Simvastatin is also known as Zocor, Synvinolin, and MK-733. Simvastatin is described in Neuvonen et al. (2008). Pharmacokinetic comparison of the potential over-the-counter statins simvastatin, lovastatin, fluvastatin and pravastatin. Clinical Pharmacokinetics 47 (7): 463-74; U.S. Pat. No. 5,393,893, issued Feb. 28, 1995; and U.S. Pat. No. 6,384,238, issued May 7, 2002, the entire contents of each of which are hereby incorporated herein in their entireties.

Lovastatin is a statin that is commercially available from Merck Sharp & Dohme Corp. (Cramlington, Northumberland, UK NE23 3JU) and Mylan Pharmaceuticals Inc. (Morgantown, W. Va. 26505). The CAS Registry number for Lovastatin is 75330-75-5. Lovastatin is also known as Monacolin K, Mevinolin, Altoprev, and Mevacor. Lovastatin is described in U.S. Pat. No. 4,231,938, issued Nov. 4, 1980 and U.S. Pat. No. 5,712,130, issued Jan. 27, 1998, the entire contents of each of which are hereby incorporated herein in their entireties.

Itavastatin is a statin that is commercially available from Kowa Pharmaceuticals America, Inc. (Montgomery, Ala. 36117). The CAS Registry number for Itavastatin is 147526-32-7. Itavastatin is also known as Pitavastatin. Itavastatin is described in U.S. Pat. No. 8,829,186, issued Sep. 9, 2014, and Kajinami et al. (2003). Pitavastatin: efficacy and safety profiles of a novel synthetic HMG-CoA reductase inhibitor. Cardiovascular drug reviews 21 (3): 199-215, the entire contents of each of which are hereby incorporated herein in their entireties.

Fluvastatin is a statin that is commercially available from Novartis Pharmaceuticals Corporation (East Hanover, New Jersey 07936). The CAS Registry number for Fluvastatin is 93957-54-1. Fluvastatin is also known as Lescol, Canef, and Vastin. Fluvastatin is described in Neuvonen et al. (2008). Pharmacokinetic comparison of the potential over-the-counter statins simvastatin, lovastatin, fluvastatin and pravastatin. Clinical Pharmacokinetics 47 (7): 463-74 and U.S. Pat. No. 8,115,013, issued Feb. 14, 2012, the entire contents of each of which are hereby incorporated herein in their entireties.

Mevastatin is a statin that is commercially available from Sigma-Aldrich Co. LLC (St Louis, Mo.). The CAS Registry number for Mevastatin is 73573-88-3. Mevastatin is also known as compactin and ML-236B. Mevastatin is described in Endo et al. (1976) ML-236A, ML-236B, and ML-236C, new inhibitors of cholesterogenesis produced by Penicillium citrinium. Journal of Antibiotics (Tokyo) 29 (12): 1346-8 and U.S. Pat. No. 7,582,464, issued Sep. 1, 2009, the entire contents of each of which are hereby incorporated herein in their entireties.

Cerivastatin is a statin. Cerivastatin sodium salt hydrate is commercially as available from Sigma-Aldrich Co. LLC (St Louis, Mo.). The ChemSpider identification number for Cerivastatin is 393588. Cerivastatin is also known as Baycol and Lipobay. Cerivastatin is described in Furberg and Pitt (2001) Withdrawal of cerivastatin from the world market. Curr Control Trials Cardiovasc Med 2:205-207 and U.S. Pat. No. 8,586,527, issued Nov. 19, 2013, the entire contents of each of which are hereby incorporated herein in their entireties.

Ezetimibe is a statin that is commercially available from Merck Sharp & Dohme Corp. (Cramlington, Northumberland, UK NE23 3JU). The CAS Registry number for Ezetimibe is 163222-33-1. Ezetimibe is also known as SCH-58235, Zetia, and Ezetrol. Ezetimibe is described in Phan et al. (2012) Ezetimibe therapy: mechanism of action and clinical update. Vasc Health Risk Manag 8: 415-27 and U.S. Patent Application Publication No. 2011/0262497, published Oct. 27, 2011, the entire contents of each of which are hereby incorporated herein in their entireties.

Numerous other statins are known in the art. Additional non-limiting examples of statins are described in U.S. Pat. No. 5,393,893, issued Feb. 28, 1995; U.S. Pat. No. 6,384,238, issued May 7, 2002; U.S. Pat. No. 6,541,511, issued Apr. 1, 2003; U.S. Pat. No. 7,166,638, issued Jan. 23, 2007; U.S. Pat. No. 6,933,292, issued Aug. 23, 2005; and U.S. Patent Application Publication No. 2008/0318920, published Dec. 25, 2008, the entire contents of each of which are hereby incorporated herein by reference.

Non-limiting examples of commercially available SSRIs include: Duloxetine, Nefazodone, Fluoxetine, and Sertraline.

Duloxetine is an SSRI that is commercially available from Eli Lilly and Company (Indianapolis, Ind. 46285). The ChemSpider identification number for Duloxetine is 54822. Duloxetine is also known as Cymbalta. Duloxetine is described in Perahia et al. (2006) Duloxetine 60 mg once daily in the treatment of milder major depressive disorder. Int. J. Clin. Pract. 60 (5): 613-20 and U.S. Pat. No. 8,269,023, the entire contents of each of which are hereby incorporated herein in their entireties.

Nefazodone is an SSRI that is commercially available from Bristol-Myers Squibb Company (Princeton, N.J. 08543). The CAS Registry number for Nefazodone is 83366-66-9. Nefazodone is also known as Dutonin, Nefadar, and Serzone (Nefazodone Hydrochloride). Nefazodone is described in Saper et al. (2001) Nefazodone for chronic daily headache prophylaxis: an open-label study. Headache 41 (5): 465-74, and U.S. Pat. No. 6,034,085, issued Mar. 7, 2000, the entire contents of each of which are hereby incorporated herein in their entireties.

Fluoxetine is an SSRI that is commercially available from Eli Lilly and Company (Indianapolis, Ind. 46285). The CAS Registry number for Fluoxetine is 54910-89-3. Fluoxetine is also known as Lilly-110140, Sarafem, and Prozac (fluoxetine hydrochloride). Fluoxetine is described in Altamura et al. (1994). Clinical Pharmacokinetics of Fluoxetine. Clinical Pharmacokinetics 26 (3): 201-214 and U.S. Pat. No. 5,166,437, issued Nov. 24, 1992, the entire contents of each of which are hereby incorporated herein in their entireties.

Sertraline is an SSRI that is commercially available from Pfizer (New York, N.Y. 10017). The CAS Registry number for Sertraline is 79617-96-2. Sertraline is also known as Zoloft (sertraline hydrochloride), and Lustral (sertraline hydrochloride). Sertraline is described in Obach et al., (2005) Sertraline is metabolized by multiple cytochrome P450 enzymes, monoamine oxidases, and glucuronyl transferases in human: an in vitro study. Drug Metab. Dispos. 33 (2): 262-70 and U.S. Pat. No. 7,186,863, issued Mar. 6, 2007, the entire contents of each of which are hereby incorporated herein in their entireties.

Numerous other SSRIs are known in the art. Additional non-limiting examples of SSRIs are described in U.S. Pat. No. 7,186,863, issued Mar. 6, 2007; U.S. Pat. No. 7,217,696, issued May 15, 2007; U.S. Pat. No. 5,104,899, issued Apr. 14, 1992; and U.S. Pat. No. 8,524,950, issued Sep. 3, 2013, the entire contents of each of which are hereby incorporated herein by reference.

Aspects of the present invention relate to compounds that inhibit the induction of MMP-1 expression by cigarette smoke or cigarette smoke extract (CSE). In some embodiments, compounds that block more than 80% and no more than 120% of cigarette or CSE induced MMP-1 expression are selected for use in treating subjects (inhibition greater than 120% would indicate baseline inhibition of MMP-1 expression unrelated to CSE). Therefore, aspects of the present invention relate to statins and SSRIs that reduce the induction of MMP-1 by cigarette smoke or CSE without reducing baseline MMP-1 expression more than 5, 10, 15, or 20%.

In come embodiments, and depending on the assay used, the percentage inhibition of the CSE/MMP-1 induction may be calculated for compounds on a per-plate basis, using the equation: % inhibition of compound=100×[1−(test well−median high-signal control)/(median high−signal control−median low-signal control)].

It will be understood by persons skilled in the art that the percent inhibition of MMP-1 induced expression may be assayed using the methods described in the Examples herein. It will also be understood that assays other than the methods exemplified herein, or variations thereof, may be used to determine the percent inhibition of the induced expression of MMP-1. Non-limiting examples of other methods for assaying the induced expression of MMP-1 (and the inhibition thereof) include quantitative real-time PCR (qPCR), Western Blot analysis, Northern Blot, and array analysis (such as microarray analysis).

Dosage Forms and Administration

Ester derivatives of compounds used in the subject invention may be generated from a carboxylic acid group in accordance with the present invention using standard esterification reactions and methods readily available and known to those having ordinary skill in the art of chemical synthesis. Ester derivatives may serve as pro-drugs that can be converted into compounds by serum esterases.

Compounds used in the methods of the present invention may be prepared by techniques well know in organic synthesis and familiar to a practitioner ordinarily skilled in the art. However, these may not be the only means by which to synthesize or obtain the desired compounds.

Compounds used in the methods of the present invention may be prepared by techniques described in Vogel's Textbook of Practical Organic Chemistry, A. I. Vogel, A. R. Tatchell, B. S. Furnis, A. J. Hannaford, P.W.G. Smith, (Prentice Hall) 5th Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

In some embodiments, a compound may be in a salt form. As used herein, a “salt” is a salt of the instant compound which has been modified by making acid or base salts of the compounds. In the case of the use of compounds for treatment of a disease, the salt is pharmaceutically acceptable. Examples of pharmaceutically acceptable salts include, but are not limited to, mineral or organic acid salts of basic residues such as amines. The term “pharmaceutically acceptable salt” in this respect, refers to the relatively non-toxic, inorganic and organic base addition salts of compounds. These salts can be prepared in situ during the final isolation and purification of a compound, or by separately reacting a purified compound in its free acid form with a suitable organic or inorganic base, and isolating the salt thus formed.

The compounds used in some embodiments of the present invention can be administered in a pharmaceutically acceptable carrier. As used herein, a “pharmaceutically acceptable carrier” is a pharmaceutically acceptable solvent, suspending agent or vehicle, for delivering the compounds to the subject. The carrier may be liquid or solid and is selected with the planned manner of administration in mind. Liposomes are also a pharmaceutically acceptable carrier. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

“Administering” compounds in embodiments of the invention can be effected or performed using any of the various methods and delivery systems known to those skilled in the art. The administering can be, for example, intranasal, intravenous, oral, intramuscular, intravascular, intra-arterial, intracoronary, intramyocardial, intraperitoneal, and subcutaneous. Aspects of the present invention relate to the nasal or oral inhalation of a compound using an inhaler. Other non-limiting examples include topical administration, or coating of a device to be placed within the subject. In some embodiments, administration is effected by injection or via a catheter.

Aspects of the present invention relate to the administration of a compound using an inhaler. In some embodiments, an amount of a compound-containing aerosol or powder is discharged into the nose or mouth of a subject using an inhaler. Non-limiting examples of inhalers are described in U.S. Pat. No. 7,900,625, issued Mar. 8, 2011; U.S. Pat. No. 5,891,419, issued Apr. 6, 1999; U.S. Pat. No. 3,456,644, issued Jul. 22, 1969; U.S. Pat. No. 6,684,879, issued Feb. 3, 2004; U.S. Pat. No. 7,448,385, issued Nov. 11, 2008; U.S. Pat. No. 8,555,878, issued Oct. 15, 2013; U.S. Pat. No. 7,073,499, issued Jul. 11, 2006; and PCT International Patent Application

Publication No. 2014/137215, published Sep. 12, 2014.

Injectable drug delivery systems may be employed in the methods described herein include solutions, suspensions, and gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

The dosage of a compound administered in treatment will vary depending upon factors such as the pharmacodynamic characteristics of the compound and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of a compound may comprise a compound alone, or mixtures of a compound with additional compounds used to treat a disease, e.g. COPD. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the eye, all using dosage forms well known to those of ordinary skill in the pharmaceutical arts.

A compound can be administered in a mixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

A compound may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

A compound may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, a compound may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain a compound and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, a compound may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

A compound may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The compounds and compositions thereof can be coated onto stents for temporary or permanent implantation into the cardiovascular system of a subject.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### EXPERIMENTAL DETAILS

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1. Translational Approach to the Treatment of COPD

Exposure to tobacco smoke is a major risk factor for chronic obstructive pulmonary disease (COPD) with the ultimate tissue destruction in emphysema resulting from an imbalance in protease/antiprotease activity. The D'Armiento laboratory has demonstrated that lung parenchymal cells in patients with emphysema express MMP-1 as opposed to smokers without the disease and through in vitro and in vivo studies we demonstrated that cigarette smoke can directly induce MMP production in epithelial cells in a MAP Kinase dependent fashion. Subsequent studies identified a novel cigarette smoke responsive (CSR) element within the promoter region of MMP-1. The upstream signaling pathway regulating MMP-1 induction by cigarette smoke was further delineated and TLR4 was identified as an important regulator of the induction of MMP-1. After the identification of the CSE in MMP-1 we used this knowledge to develop a novel mammalian cell-based assay to screen for inhibitors to the smoke induced MMP-1 pathway by transfecting a human cell line (HEK 293T) with a vector containing a luciferase reporter gene under the control of the MMP-1 promoter. Using this novel cell based system we screened an NIH library of compounds and identify novel compounds that exhibited strong activity in our assay. This screening has led to several candidate molecules we are pursuing for the treatment of emphysema.

Studies are conducted to elucidate the role of these compounds in treating emphysema.

### Example 2. Inhibitors of Cigarette Smoke Induced MMP-1 Production (SSRI)

Preliminary Studies

The aim of this study is to develop molecules that can modulate both the transcriptional induction of MMPs and the inflammatory cascade induced by cigarette smoke. As a preliminary essential step in this proposed study, a mammalian cell-based assay was developed based on transfection of a human cell line (HEK 293T) with a vector containing a luciferase reporter gene under the control of the MMP-1 promoter. This method is based on the fact that the MMP-1 promoter contains a specific cigarette smoke responsive element (CRE) (Golovatch et al., 2009).

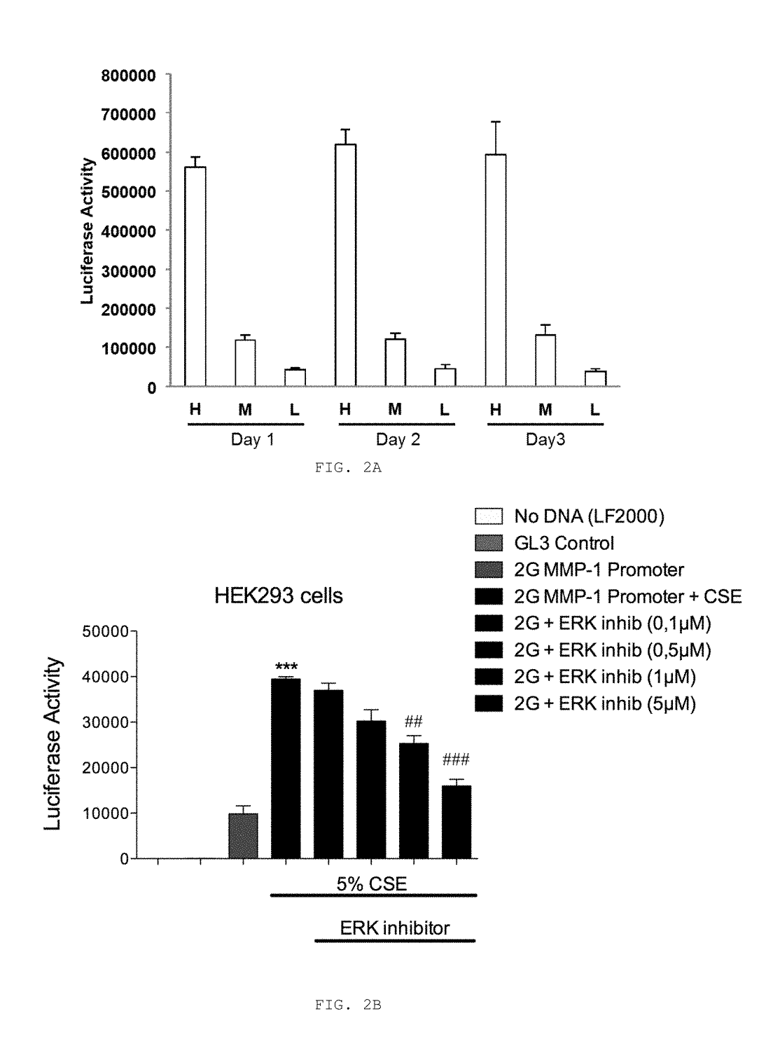

Utilizing the MMP-1 promoter, an MMP-1/pGL3 luciferase reporter vector was prepared and transfected into cells. The assay was developed by treating transfected cells with cigarette smoke, tested for reproducibility and inhibition with MAPKinase inhibitors that were known to block smoke induced MMP-1 expression (FIGS. 2A and 2B) (Mercer et al., 2004). The assay was shown to be stable over several days in separate batches. The cigarette smoke induction is MAPKinase dependent therefore the dose dependency of the assay was demonstrated using a MAPKinase inhibitor.

Pilot Screen of Small Molecules from the NIH Clinical Collection and their Validation

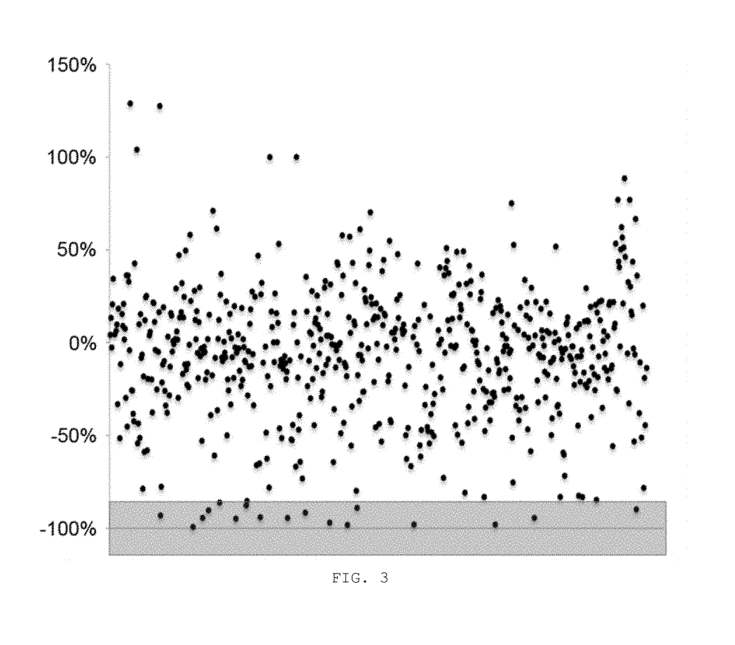

Utilizing the developed assay above, the effect of a collection of 727 structurally diverse small molecules obtained from the NIH clinical collection was tested. The compounds in this clinical set have all been tested and utilized in humans for various indications. These molecules, dissolved in DMSO, were all tested at the concentration of 10 μM for their capacity to modulate MMP-1 smoke induced transcriptional activation. The percentage inhibition of the CSE/MMP-1 induction was calculated for each tested compound on a per-plate basis, using the equation: % inhibition of compound=100×[1−(test well−median high-signal control)/(median high-signal control−median low-signal control)]. Compounds that block more than 80% and no more than 120% of CSE induced MMP-1 expression are considered as initial hit compounds (Inhibition greater than 120% would indicate baseline inhibition of MMP-1 expression unrelated to CSE). As shown in FIG. 3, fifteen of the tested returned Luciferase activity to baseline. As expected, none of the compounds exhibited cytotoxic activity, as assayed by the CellTiter-Glo (Promega Corp.; data not shown).

At this point 10 promising compounds were selected, and classified in two major categories as follows:
A. Statins (Simvastatin, Lovastatin, Fluvastatin, Mevastatin, Cerivastatin, Ezetimibe),
B. Selective Sertonin Reuptake Inhibitors (Duloxetine, Nefazodone, Fluoxetine, sertraline,)

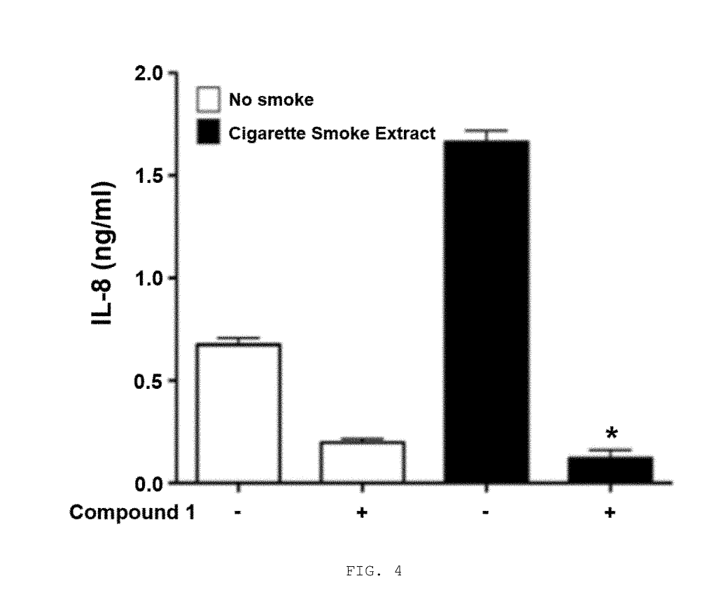

A purpose of these studies was to identify compounds that not only block transcription of MMP-1 but other MMPs and cytokines important in the pathogenesis of COPD (Decramer et al., 2012). Therefore, after completion of the above studies involving the primary screening campaign the activity and potency of validated hits was confirmed in secondary assays to assess their effect on the up regulation of IL-8 (CXCL8) in small airway epithelial cells (SAECs) treated with CSE (FIG. 4). IL-8 is a major chemokine increased in the sputum of COPD patients and correlates with the number of neutrophils present in the lung (Barnes et al., 2004). The neutrophil is a major inflammatory cell present as a result of smoke exposure (Stockley et al., 2009) and is thought to be recruited through the induction of IL-8 by cigarette smoke (Moon et al., 2013). Therefore, identifying mechanisms to block production of IL-8 will prevent the influx of neutrophils and limit inflammation secondary to cigarette smoke. These assays, based on established protocols for treatment of SAECs (Lonza, Walkersville, Md.) with cigarette smoke extract (Mercer et al., 2004), were performed in the same format optimized for the initial screen.

The In Vivo Effect of these Compounds on an Animal Model of COPD

After confirming the in vitro activity of the compounds within the two catagories described above, rabbits were then treated under smoke exposure conditions with Duloxetine and examined the effect on MMP-1 expression. Rabbits were stratified into one of four groups (Unexposed, treated with vehicle, Smoke exposed, treated with vehicle, Unexposed, treated with Duloxetine and smoke exposed treated with Duloxetine. Animals are maintained on room air or smoke exposed for 16 weeks and then sacrificed. Lung lavage is taken, lungs are fixed and sectioned and protein homogenate from lungs frozen.

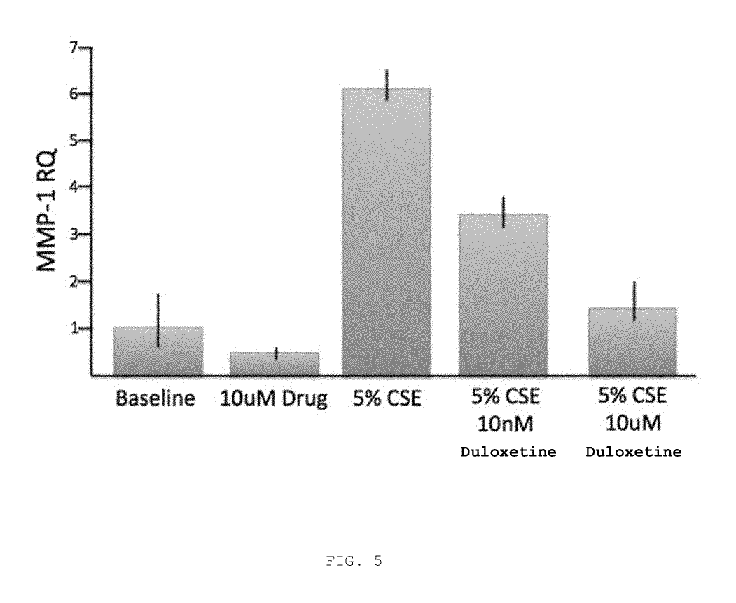

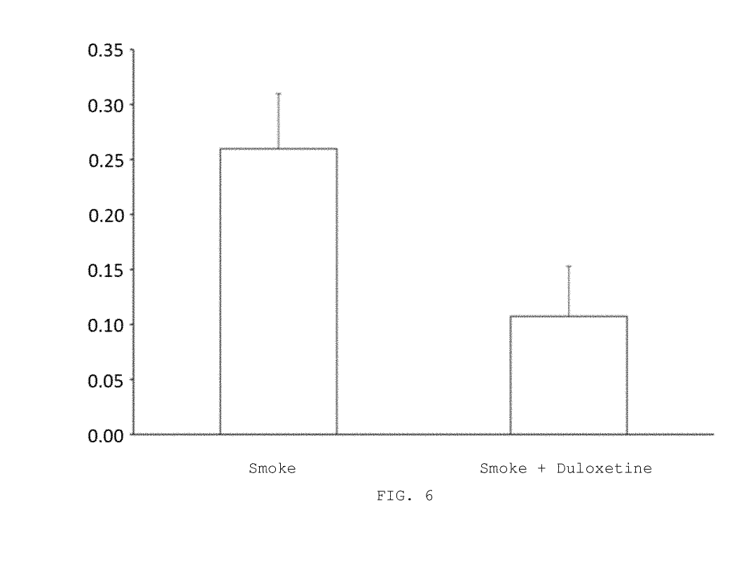

The lung lavage from the rabbits indicates that there in an increase in MMP-1 protein expression in rabbits exposed to cigarette smoke and the addition of Duloxetine blocked the increase in MMP-1 protein suggesting that the compound blocked the induction of MMP-1 caused by cigarette smoke (FIG. 5).

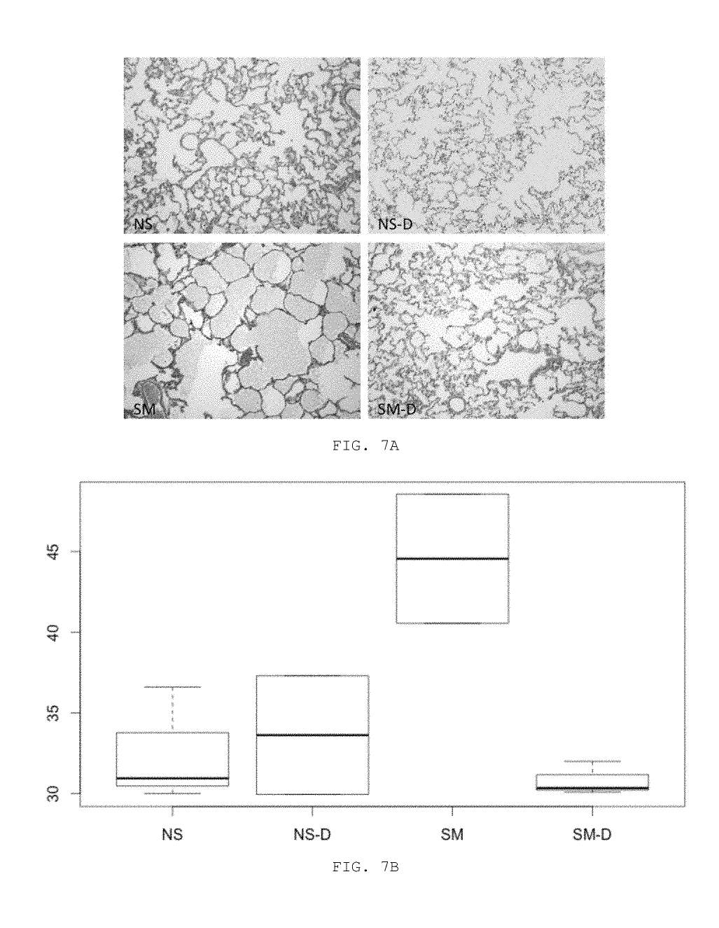

Histological analysis of the lungs demonstrated that rabbits exposed to smoke developed emphysema and when treated with duloxetine emphysema was attenuated (FIGS. 7A, 7B).

Conclusion

These results indicate that the cells treated with SSRI (duloxetine) exposed to cigarette smoke inhibited MMP-1 expression. These data also demonstrate that the compound can modulate the expression of cigarette smoke induced MMP-1 expression and protect from the development of emphysema.

### Example 3

An experiment was performed to determine if Fluoxetine (an SSRI) could block transcription of MMP1 and cell signaling in cigarette smoke exposed SAE cells. Additionally, studies were undertaken to ensure that Fluoxetine was absorbed into the lung when delivered by gavage and inhalation.

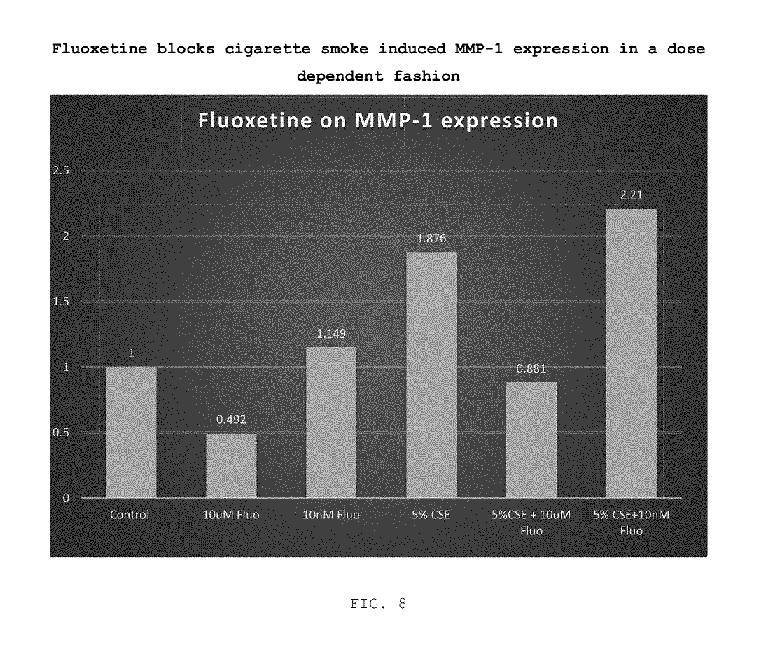

In the first experiment, SAE cells were treated with 5% CSE, 10 nM Fluoxitine, 10 uM Fluoxitine, and combinations of 5% CSE and Fluoxitine (FIG. 8). CSE induces MMP1 expression and when treated with Fluoxetine and CSE MMP-1 induction was attenuated (p<0.05). Data is presented as mean+standard error.

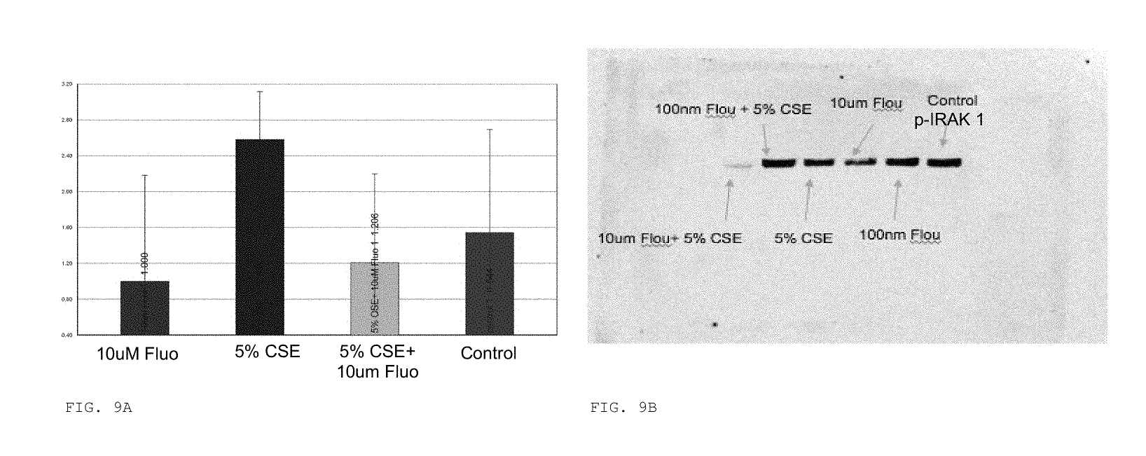

SAE cells treated with 5% CSE and 10 uM fluoxetine (Fluo) were shown to exhibit down regulation of TLR-4 receptor expression (FIG. 9A). Further, Western blot analysis of the treated cells demonstrates that the phosphorylation of IRAK (downstream target of TLR-4 signaling pathway) was suppressed by Fluoxetine (FIG. 9B).

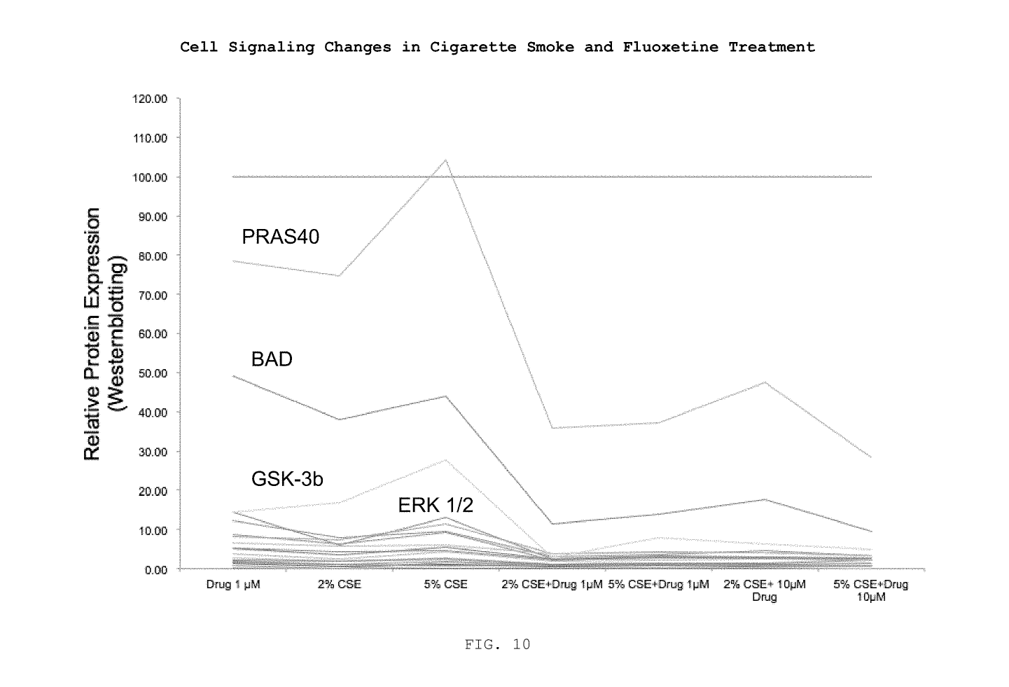

Next, cells treated with CSE were compared to cells treated with CSE combined with fluoxetine using a protein array analysis (FIG. 10). Three transcription factors were found to increase with CSE treatment and when pretreated with Fluoxetine these factors return to baseline. PRAS40, BAD and GSK-3b are increased with cigarette smoke treatment and return to baseline upon Fluoxetine treatment.

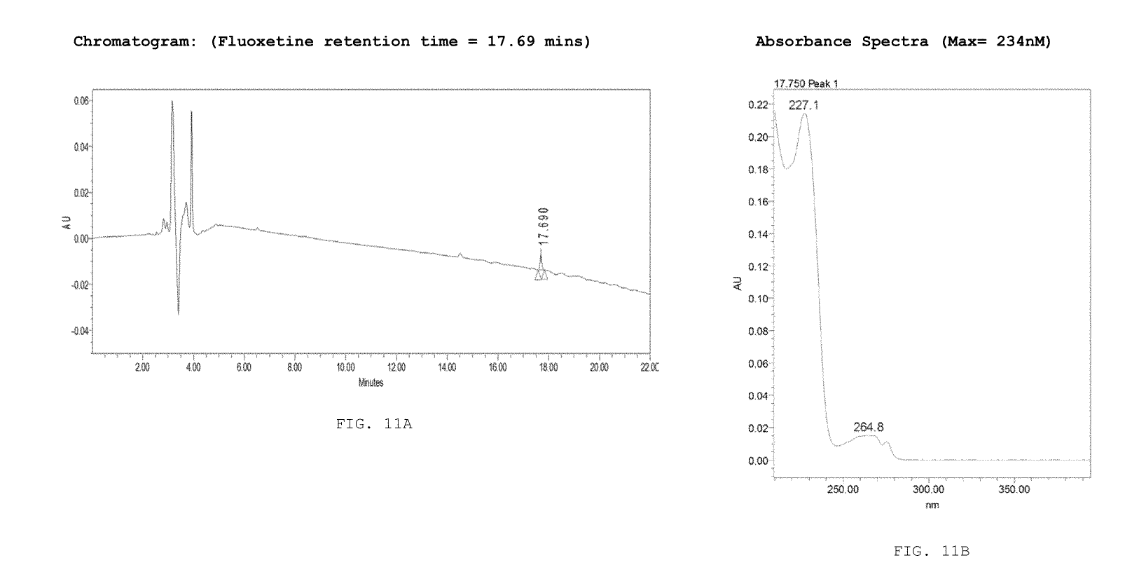

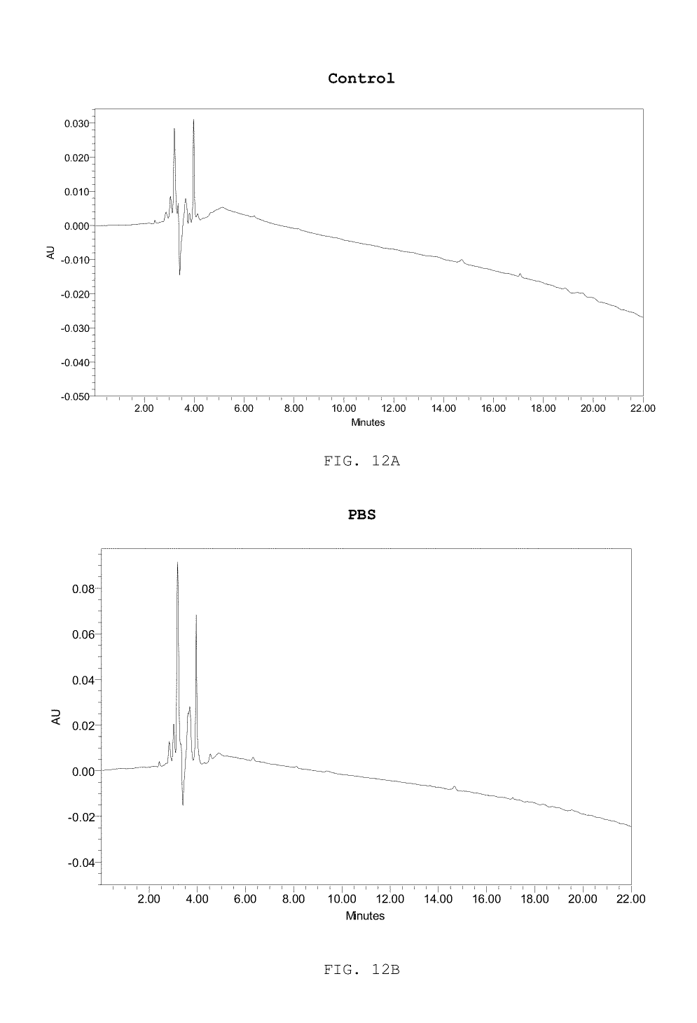

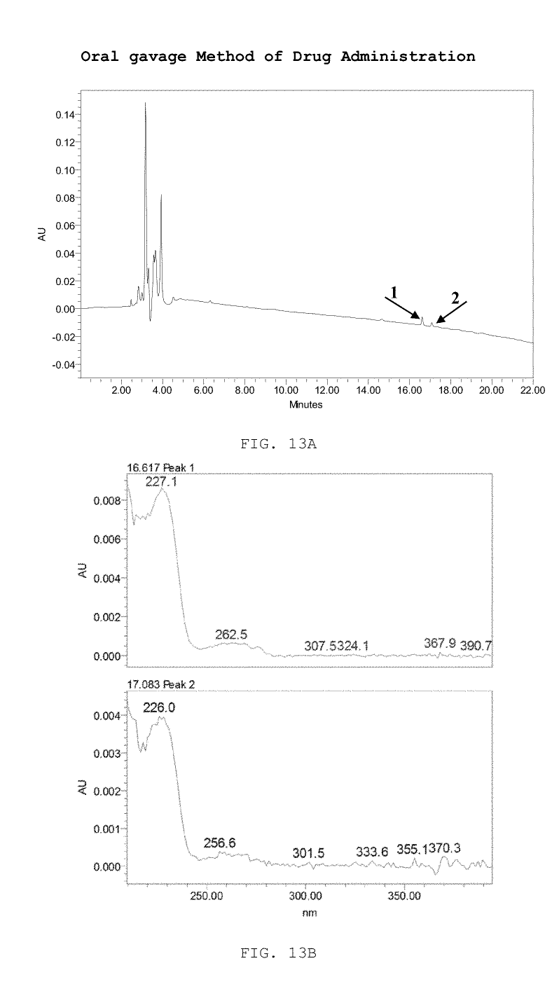

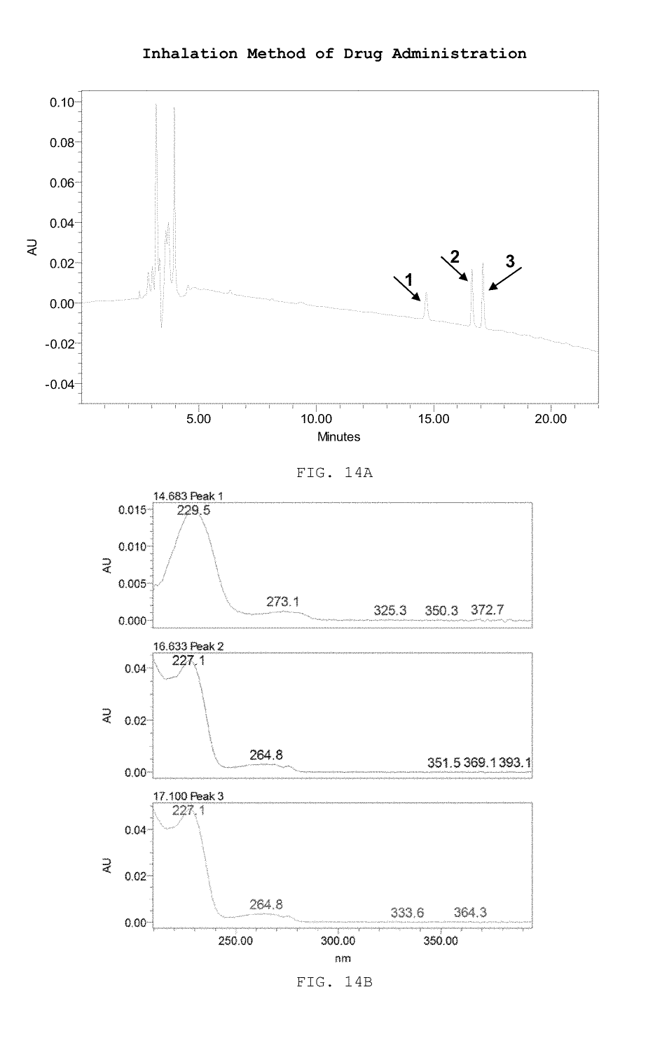

Studies were also undertaken to quantify fluoxetine in lung samples. FIGS. 11A and 11B demonstrate the standard chromatographic characteristics of Fluoxetine and FIGS. 12A and 12B demonstrate the standard chromatographic characteristics of a control (no drug administration) and PBS, respectively. In order to determine whether fluoxetine is absorbed in the lung, fluoxetine was delivered either through oral gavage or inhaled. Animals were given PBS, 10 mg/kg oral, gavage of fluoxetine (FIGS. 13A and 13B), and 10 mg/kg of inhaled Fluoxetine (FIGS. 14A and 14B). Mice were sacrificed, lungs lyophilized and protein homogenate prepared for chromatographic analysis. Both oral and inhaled delivery of Fluoxetine resulted in the detection of Fluoxetine in the lung of mice. Inhalation resulted in higher levels of Fluoxetine within the lung.

Oral gavage of fluoxetine yielded two compounds showing chromatographic peaks at similar retention times and absorbance spectra that suggested the occurrence of a metabolite of fluoxetine, an isomer or enantiomer of the compound (FIGS. 13A and 13B). Oral gavage of fluoxetine yielded lower quantifiable concentrations than nebulized administration of the drug.

Following drug inhalation, three compounds could be identified in lung samples, where two of the compounds had almost identical absorbance spectra; the third compound retained a more polar characteristic eluting at 14.7 mins (FIGS. 14A and 14B).

Detailed quantification results for fluoxetine inhaled or orally administered are found in the tables below.

TABLE 1
Administration of Fluoxetine through Oral Gavage

| | Retention Time | Area | | mM | mg/ml | ng/ml |
|---|---|---|---|---|---|---|
| Replicate 1 | 17.08010655 | 12906.1476 | | 0.003711878 | 0.001283493 | 1283.493254 |
| | 16.6131189 | 27449.95881 | | 0.005789566 | 0.002001916 | 2001.915974 |
| Replicate 2 | 17.08063126 | 12631.40873 | | 0.00367263 | 0.001269922 | 1269.921939 |
| | 16.61380581 | 32793.07907 | | 0.006552868 | 0.002265851 | 2265.850849 |
| Replicate 3 | 17.08660679 | 14782.81571 | | 0.003979974 | 0.001376195 | 1376.195297 |
| | 16.61993572 | 33850.94473 | | 0.006703992 | 0.002318106 | 2318.10639 |
| | | | Average (17.08) | 0.003788161 | 0.00130987 | 1309.870163 |
| | | | SD (17.08) | 0.00016727 | 5.78387E−05 | 57.83867771 |
| | | | Average (16.6) | 0.006348809 | 0.002195291 | 2195.291071 |
| | | | SD (16.6) | 0.000490178 | 0.000169494 | 169.4936767 |

TABLE 2
Administration of Fluoxetine Through Inhalation

| | Injection # | Retention Time | Area | | mM | mg/ml | ng/ml |
|---|---|---|---|---|---|---|---|
| Replicate 1 | 3 | 17.0888794 | 172094.4234 | | 0.02645306 | 0.009146939 | 9146.939256 |
| | 2 | 16.62415789 | 148723.6065 | | 0.023114372 | 0.007992488 | 7992.487673 |
| | 1 | 14.67573133 | 103467.6833 | | 0.01664924 | 0.005756974 | 5756.974369 |
| Replicate 2 | 1 | 14.67975811 | 94922.53266 | | 0.015428505 | 0.005334868 | 5334.868343 |
| | 2 | 16.63809468 | 146811.073 | | 0.022841153 | 0.007898014 | 7898.013983 |
| | 3 | 17.10260277 | 167626.8324 | | 0.025814833 | 0.008926253 | 8926.253025 |
| Replicate 2 | 3 | 17.10524028 | 170318.3142 | | 0.026199331 | 0.009059205 | 9059.204534 |
| | 2 | 16.64006528 | 151315.2219 | | 0.023484603 | 0.008120506 | 8120.506072 |
| | 1 | 14.67979678 | 104234.9551 | | 0.016758851 | 0.005794875 | 5794.875404 |
| | | | | Average (17.08) | 0.026155741 | 0.009044132 | 9044.132272 |
| | | | | SD (17.08) | 0.000321339 | 0.000111112 | 111.1124791 |
| | | | | Average (16.6) | 0.02314671 | 0.008003669 | 8003.669243 |
| | | | | SD (16.6) | 0.000322941 | 0.000111667 | 111.6667051 |
| | | | | Average (14.6) | 0.016278865 | 0.005628906 | 5628.906039 |
| | | | | SD (16.6) | 0.00073847 | 0.000255348 | 255.3482855 |

Results

The experiments demonstrated that MMP1 induction by cigarette smoke in SAEC cells could be attenuated by treatment with Fluoxetine (FIG. 8). These studies demonstrate that Fluoxetine behaves in a similar fashion as duloxetine in vitro. The ability of Fluoxetine to act on the TLR4 signaling pathway was then examined. FIGS. 9A and 9B demonstrate that while CSE increases TLR4 signaling this pathway is attenuated when cells are treated with Fluoxetine.

Finally, studies were carried out in vivo to determine if Fluoxetine delivered both orally and through inhalation was present within the lung of mice. As seen in FIGS. 11A-14B Fluoxetine was present within the lung under both delivery methods with inhalation revealing higher levels.

In conclusion these results indicate that cells treated with SSRI (Fluoxetine) exposed to cigarette smoke inhibited MMP-1 expression. Furthermore, Fluoxetine is absorbed in the lung when delivered through gavage or inhaled.

Discussion

Chronic obstructive pulmonary disease (COPD) is the third leading cause of death in the United States (Podowski et al. (2012); Mannino et al. (2007)) with tobacco smoke the key etiologic agent of this disease process; the inflammatory response to inhaled cigarette smoke and other noxious particles (Global Initiative for Chronic Obstructive Lung Disease, 2011; Global Initiative for chronic obstructive Lung Disease., 2007) is thought to be a primary initiator of the disease. COPD is characterized by progressive airflow limitation that is not fully reversible. A spectrum of pathological findings are observed in COPD ranging from inflammation of the larger airways (termed chronic bronchitis), remodeling of the small airways, and parenchymal tissue destruction with airspace enlargement (defined as emphysema) (Global Initiative for Chronic Obstructive Lung Disease, 2011; Global Initiative for chronic obstructive Lung disease., 2007). In addition, COPD contributes to systemic manifestations affecting skeletal muscles, bone and the cardiovascular system (Yoshida and Tuder (2007); Celli et al. (2006)). Despite the heterogeneity of COPD, the small airway walls in the emphysematous lung consistently demonstrate persistent inflammation with mononuclear phagocytes that play a major role in the inflammatory response (Shan et al. (2009); Shaykhiev et al. (2009)).
Chronic obstructive pulmonary disease (COPD) is a progressively worsening lung disease that is characterized by disrupted airflow (Decramer et al. (2012)).
Currently, approximately a third of a billion people suffer from COPD.
COPD is one of the top 10 leading causes of death, according to the WHO (World Health Organization (2013)).
COPD is most often caused by cigarette smoke (Decramer et al. (2012)).
Constant exposure to cigarette smoke eventually causes COPD to develop into emphysema (Rabe et al. (2007)).
Matrix metalloproteinase-1 (MMP-1) play an important role in the development of COPD (D'Armiento et al. 1992).
MMP-1 contains a cigarette smoke response element in its promoter, indicating that cigarette smoke promotes COPD and emphysema via MMP-1 expression (Mercer et al. (2009)).

This Technology
Using a luciferase based reporter assay to look for compounds that bind to the MMP-1 promoter region, two classes of drugs were found to inhibit MMP-1.
Selective serotonin reuptake inhibitors (SSRIs) and statins were both capable of inhibiting MMP-1 at low concentrations.
In vitro data showed that MMP-1 expression decreased with one of the compounds (Duloxetine).

Discussion

Cigarette smoke intake results in a number of comorbidities, including chronic obstructive pulmonary disease (COPD)/emphysema, a debilitating lung disease that afflicts millions of smokers. A family of proteins known as matrix metalloproteinases, or MMPs, regulates the progression of COPD/emphysema. Without wishing to be bound by any scientific theory, since cigarette smoke can directly bind to the promoter region of MMP-1, one of the proteins in the MMP family, and induce expression, a therapeutic strategy is to inhibit MMP-1 transcription to potentially delay or halt the development of COPD/emphysema in smokers.

Using an in vitro screening assay that targets the response element, this technology has identified two drug classes that decrease MMP-1 transcription. The classes, SSRIs and statins, inhibited MMP-1 mRNA at low concentrations, making them ideal therapeutic candidates. Identification of these small molecules may potentially provide a viable means of treating COPD, as well as other highly prevalent pathologies that respond to these molecules, such as arthritis and atherosclerosis.

COPD/emphysema is a highly prevalent disease. While it is clearly established that cigarette smoke is the principal cause of COPD, the mechanism by which cigarette smoke exposure leads to destruction of the lung architecture as seen in emphysema is unknown. The D'Armiento laboratory was the first to demonstrate a direct role for MMPs in emphysema causation through the generation of several transgenic mouse lines that express human MMP-1 in lung epithelial cells. Recent studies in the D'Armiento laboratory have shown that cigarette smoke can induce expression of MMP-1 in resident lung cells in emphysema and have identified a cigarette smoke responsive element in the MMP-1 promoter region.

In order to identify molecules that can modulate the transcriptional activity of MMP-1 induced by cigarette smoke, a mammalian cell line-based transfection assay in a 96-well format was developed that can easily be implemented for HTS. The method is based on transfection of a human cell line (HEK 293T) with a vector containing a luciferase reporter gene, which is under the control of the MMP-1 promoter. Then, the effect of a small collection of 727 structurally diverse small molecules was tested on the MMP-1 transcriptional activity. The molecules were obtained from NIH clinical collection.

Through this pilot screening, two classes of drugs that can prevent the expression of MMP-1 induced by cigarette smoke were identified. The one is selective serotonin reuptake inhibitors (SSRIs) and the other is Statins. Both drugs could inhibit the MMP-1 expression less than 10 nM concentrations. In addition, one of the compounds, Duloxetine ((+)-(S)—N-Methyl-3-(naphthalen-1-yloxy)-3-(thiophen-2-yl) propan-1-amine; Cymbalta®) was studied and it could block the MMP-1 expression at the concentration of 10 nM in cell culture system as well as rabbit cigarette smoke model by ELISA of lung homogenate (20 weeks of cigarette smoke and giving the compound in the last four weeks).

Apart from its role in emphysema formation described above, MMP-1 has been implicated in several pathological processes, including tumor invasion, arthritis, skin repair and atherosclerotic plaque rupture. Therefore, the small molecules identified in this study will have wide applicability for the treatment of various diseases.

The present invention provides compounds and methods for preventing the destruction of lung in emphysema. Additionally, compounds of the invention may be used prophylactically, to prevent the development of COPD/emphysema. Because MMP-1 is involved in arthritis, the compounds can also be used to treat arthritis. MMP-1 is also implicated in atherosclerosis, and therefore the compounds can also be used to treat arthritis.

Emphysema is a debilitating lung condition that affects millions of smokers. Cigarette smoke may cause emphysema by directly activating MMP-1 expression, a matrix metalloproteinase protein involved in promoting the disease. This technology has identified two classes of drugs that inhibit MMP-1 expression. With the discovery of these inhibitors, the technology may prevent millions of smokers from developing emphysema.

The present invention is unique since it relates to the identification of compounds directly targeting the pathogenic processes responsible for lung destruction in COPD and not simply treating the symptoms of disease. Initial studies have identified compounds that block smoke induced protease production and inflammation. Therefore, the use of such compounds will be exclusive in the field of COPD with the ability to actually target two processes known to be important in actively degrading and damaging the lung secondary to cigarette smoke (Barnes, 2003). These compounds would therefore benefit not only severely affected COPD patients but potentially target all patients with COPD to stabilize disease and protect the lung from further destruction.

### REFERENCES

Barnes P J. Mediators of chronic obstructive pulmonary disease. Pharmacol Rev. 2004; 56(4):515-48.
Barnes P. New concepts in chronic obstructive pulmonary disease. Annu Rev Med. 2003; 54, 113-29.
Centers for Disease C, and Prevention. Current cigarette smoking among adults—United States, 2011. MMWR Morb Mortal Wkly Rep. 2012; 61(44):889-94 (“CDC, 2012”)
Celli B R. Roger s. Mitchell lecture. Chronic obstructive pulmonary disease phenotypes and their clinical relevance. Proc Am Thorac Soc. 2006; 3(6):461-5.
Chronic Obstructive Pulmonary Disease (COPD) Fact Sheet. (2014) www.lung.org/lung-disease/copd/resources/facts-figures/COPD-Fact-Sheet.html. Accessed Dec. 3, 2014
Chronic Obstructive Pulmonary Disease (COPD) Market to 2019. (2013) www.gbiresearch.com/report-store/market-reports/therapy-analysis/chronic-obstructive-pulmonary-disease-(copd)-market-to-2019-highly-priced-new-combination-products-forecast-to-capture-signif. Accessed Dec. 3, 2014
D'Armiento J, Dalal S S, Okada Y, Berg R A, Chada K. (1992). Collagenase expression in the lungs of transgenic mice causes pulmonary emphysema. Cell. 71:955-961.
Decramer M, Janssens W, Miravitlles M. (2012) Chronic obstructive pulmonary disease. The Lancet 379:1341-1351.
Fabbri L M, Beghe B, Yasothan U, and Kirkpatrick P. Roflumilast. Nature reviews Drug discovery. 2010; 9(10):761-2.
From the Centers for Disease Control and Prevention. Prevalence of current cigarette smoking among adults and changes in prevalence of current and some day smoking—United States, 1996-2001. JAMA. 2003; 289(18):2355-6 (“CDC 2003”)
Global Initiative for Chronic Obstructive Lung Disease (GOLD); 2013.
Global Initiative for Chronic Obstructive Lung Disease, 2011. Global strategy for the diagnosis, management, and prevention of chronic obstructive pulmonary disease.
Global Initiative for chronic obstructive Lung disease., 2007. Global strategy for the diagnosis, management, and prevention of chronic obstructive pulmonary disease.
Golovatch P, Mercer B A, Lemaitre V, Wallace A, Foronjy R F, and D'Armiento J. Role for cathepsin K in emphysema in smoke-exposed guinea pigs. Exp Lung Res. 2009; 35(8):631-45.
Hanania N A, and Marciniuk D D. A unified front against COPD: clinical practice guidelines from the American College of Physicians, the American College of Chest Physicians, the American Thoracic Society, and the European Respiratory Society. Chest. 2011; 140(3):565-6.
Mannino D M, Buist A S. Global burden of COPD: risk factors, prevalence, and future trends. Lancet. 2007; 370(9589):765-73.
Mercer B A, Wallace A M, Brinckerhoff C E, and D'Armiento J M. Identification of a cigarette smoke-responsive region in the distal MMP-1 promoter. Am J Respir Cell Mol Biol. 2009; 40(1):4-12.
Mercer B A, Kolesnikova N, Sonett J, and D'Armiento J. Extracellular regulated kinase/mitogen activated protein kinase is up-regulated in pulmonary emphysema and mediates matrix metalloproteinase-1 induction by cigarette smoke. J Biol Chem. 2004; 279(17):17690-6.
Minino A M, Murphy S L, Xu J, and Kochanek K D. Deaths: final data for 2008. Natl Vital Stat Rep. 2011; 59(10):1-126.
Moon H G, Zheng Y, An C H, Kim Y K, and Jin Y. CCN1 Secretion Induced by
Cigarette Smoking Extracts Augments IL-8 Release from Bronchial Epithelial Cells. PLoS One. 2013; 8(7):e68199.
Podowski M, Calvi C, Metzger S, Misono K, Poonyagariyagorn H, Lopez-Mercado A, et al. Angiotensin receptor blockade attenuates cigarette smoke-induced lung injury and rescues lung architecture in mice. J Clin Invest. 2012; 122(1):229-40. PubMed Central PMCID: PMC3248282.
Rabe K F, Hurd S, Anzueto A, Barnes P J, et al. (2007). Global Strategy for the Diagnosis, Management, and Prevention of Chronic Obstructive Pulmonary Disease. Am J Respir Crit Med 176:532-55.
Shan M, Cheng H F, Song L Z, Roberts L, Green L, Hacken-Bitar J, et al.
Lung myeloid dendritic cells coordinately induce TH1 and TH17 responses in human emphysema. Sci Transl Med. 2009; 1(4):4ra10.
Shaykhiev R, Krause A, Salit J, Strulovici-Barel Y, Harvey B G, O'Connor T P, et al. Smoking-dependent reprogramming of alveolar macrophage polarization: implication for pathogenesis of chronic obstructive pulmonary disease. J Immunol. 2009; 183(4):2867-83. PubMed Central PMCID: PMC2873685.
Stockley R A, Mannino D, and Barnes P J. Burden and pathogenesis of chronic obstructive pulmonary disease. Proc Am Thorac Soc. 2009; 6(6):524-6.
World Health Organization. (2013, July) The Top 10 Causes of Death. Retrieved from who.int/mediacentre/factsheets/fs310/en/.
Yoshida T, Tuder R M. Pathobiology of cigarette smoke-induced chronic obstructive pulmonary disease. Physiol Rev. 2007; 87(3):1047-82.

What is claimed is:

1. A method of prophylaxis for, or for treating, chronic obstructive pulmonary disease (COPD), cancer, arthritis, skin damage, or atherosclerotic plaque rupture in a subject in need thereof, which comprises administering to the subject i) a statin or ii) a selective serotonin reuptake inhibitor (SSRI) in an amount that is effective to treat the COPD, cancer, arthritis, skin damage, or atherosclerotic plaque rupture.

2. The method of claim 1, for treatment of a subject who has been diagnosed with COPD, cancer, arthritis, skin damage, or atherosclerotic plaque rupture.

3. The method of claim 2, for treating COPD.

4. The method of claim 1, for prophylactic treatment of a subject for COPD.

5. The method of any one of claims 1-4, wherein the amount of the statin or the SSRI is effective to improve pulmonary function in the subject compared to i) the subject before administration of the amount of the statin or the SSRI or ii) a corresponding subject who has not been administered the amount of the statin or the SSRI.

6. The method of any one of claims 1-4, wherein the amount of the statin or the SSRI is effective to reduce pulmonary inflammation in the subject compared to i) the subject before administration of the amount of the statin or the SSRI or ii) a corresponding subject who has not been administered the amount of the statin or the SSRI.

7. The method of claim 6, wherein reduced pulmonary inflammation in the subject comprises
a) reduced expression of at least one cytokine or
b) a reduced number of neutrophils in the lungs of the subject.

8. The method of claim 7, wherein reduced pulmonary inflammation in the subject comprises a reduced expression of interleukin 8 (IL-8) in the lungs of the subject.

9. The method of any one of claims 1-8, wherein treating the subject comprises reducing the expression of at least one protease in the lungs of the subject compared to i) the subject before administration of the amount of the statin or the SSRI or ii) a corresponding subject who has not been administered the amount of the statin or the SSRI.

10. The method of claim 9, wherein the at least one protease is at least one matrix metalloproteinase (MMP).

11. The method of claim 10, wherein the at least one MMP comprises at least MMP-1, MMP-2, MMP-9, MMP-12 or MMP-13.

12. The method of any one of claims 1-11, wherein the COPD comprises emphysema.

13. The method of claim 12, wherein the amount of the statin or the SSRI is effective to slow, halt, or reverse the progression of emphysema in the subject.

14. The method of any one of claims 1-13, wherein the statin or the SSRI is capable of reducing cigarette smoke-induced or cigarette smoke extract (CSE)-induced MMP-1 expression without causing cytotoxicity.

15. The method of any one of claims 1-14, wherein the statin or the SSRI is capable of reducing cigarette smoke-induced or CSE-induced MMP-1 expression with an IC50 equal to or less than 1 μM.

16. The method of any one of claims 1-15, wherein the statin or the SSRI is capable of reducing cigarette smoke-induced or CSE-induced MMP-1 expression by 80-120%, wherein the level of MMP-1 expression in the absence of cigarette smoke or CSE induction is 100%.

17. The method of any one of claims 1-16, wherein the statin or the SSRI is capable of reducing MMP-1 or IL-8 expression in small airway epithelial cells (SAECs) contacted with cigarette smoke or CSE.

18. The method of any one of claims 1-17, wherein the statin or the SSRI is capable of reducing expression of TLR-4 receptor, PRAS40, BAD or GSK-3b, or reducing IRAK phosphorylation in SAECs in the subject.

19. The method of claim 17 or 18, wherein the expression is reduced by 80-120%, wherein the baseline level of expression is 100%.

20. The method of any one of claims 1-19, wherein the statin or the SSRI is an organic compound having a molecular weight less than 1000 Daltons, a DNA aptamer, an RNA aptamer, or a polypeptide.

21. The method of claim 20, wherein the statin or the SSRI is an organic compound having a molecular weight less than 1000 Daltons.

22. The method of any one of claims 1-21, wherein a statin is administered to the subject.

23. The method of claim 22, wherein the statin is Simvastatin, Lovastatin, Itavastatin, Fluvastatin, Mevastatin, Cerivastatin or Ezetimibe, or a pharmaceutically acceptable salt or ester thereof.

24. The method of any one of claims 1-23, wherein the statin is a compound that
a) is in a clinical trial;
b) is approved for use in human subjects; or
c) was previously approved for use in human subjects but whose approval was subsequently withdrawn.

25. The method of any one of claims 1-21, wherein an SSRI is administered to the subject.

26. The method of claim 25, wherein the SSRI is Duloxetine, Nefazodone, Fluoxetine or Sertraline, or a pharmaceutically acceptable salt or ester thereof.

27. The method of any one of claim 1-21, 25 or 26, wherein the SSRI is a compound that
a) is in a clinical trial;
b) is approved for use in human subjects; or
c) was previously approved for use in human subjects but whose approval was subsequently withdrawn.

28. The method of any one of claims 1-27, wherein the subject is a mammalian subject.

29. The method of any one of claims 1-28, wherein the subject is a human subject.

30. The method of any one of claims 1-29, wherein the subject is or was a cigarette smoker.

31. The method of any one of claims 1-30, wherein the COPD is caused by chronic cigarette smoking.

32. The method of any one of claims 1-31, wherein the statin or the SSRI is administered to the subject as an aerosol.

33. The method of claim 32, wherein the statin or the SSRI is administered to the subject using an inhaler.

34. The method of any of claims 1-33, wherein the statin or the SSRI is administered to the subject in a dose of between 0.1 mg/kg to 2 mg/kg.

35. The method of any of claims 1-34, wherein the statin or the SSRI is administered to the subject in a dose of about 0.8 mg/kg.

36. The method of any one of claim 1, 2 or 19-29, for treating skin damage.

37. The method of any one of claim 1, 2, 20-30 or 36, wherein administering the statin or the SSRI to the subject comprises topically applying the statin or the SSRI to the subject's skin.

38. The method of any one of claims 1-37, wherein the amount of the statin or the SSRI is effective to reduce the expression of at least one cytokine or at least one protease in the subject compared to i) the subject before administration of the amount of the statin or the SSRI or ii) a corresponding subject who has not been administered the amount of the statin or the SSRI.

39. The method of any one of claims 1-38, wherein the subject is
a) a COPD-drug, cancer-drug, arthritis-drug, skin damage-drug, or atherosclerotic plaque rupture-drug naïve subject;
b) a statin naïve subject; or
c) an SSRI naïve subject.

40. A composition for use in prophylaxis for, or in treating, chronic obstructive pulmonary disease (COPD), cancer, arthritis, skin damage, or atherosclerotic plaque rupture in a subject in need thereof which comprises i) a statin or ii) a selective serotonin reuptake inhibitor (SSRI).

41. The composition of claim 39, wherein the subject is
a) a COPD-drug, cancer-drug, arthritis-drug, skin damage-drug, or atherosclerotic plaque rupture-drug naïve subject;
b) a statin naïve subject; or
c) an SSRI naïve subject.

42. Use of i) a statin or ii) a selective serotonin reuptake inhibitor (SSRI) for the manufacture of a medicament for the treatment of chronic obstructive pulmonary disease (COPD), cancer, arthritis, skin damage, or atherosclerotic plaque rupture in a subject in need thereof.

43. The use of claim 42, wherein the treatment is prophylactic treatment.

44. The use of claim 42 or 43, wherein the subject is
a) a COPD-drug, cancer-drug, arthritis-drug, skin damage-drug, or atherosclerotic plaque rupture-drug naïve subject;
b) a statin naïve subject; or
c) an SSRI naïve subject.

45. An inhaler containing a statin or an SSRI.

46. The inhaler of claim 45, for use in treating a subject afflicted with chronic obstructive pulmonary disease (COPD).

47. The inhaler of claim 45 or 46, wherein the subject is
a) a COPD-drug, cancer-drug, arthritis-drug, skin damage-drug, or atherosclerotic plaque rupture-drug naïve subject;
b) a statin naïve subject; or
c) an SSRI naïve subject.
